(19) United States
(12) Patent Application Publication
(10) Pub. No.: US 2016/0317268 A1
(43) Pub. Date: Nov. 3, 2016

(54) STORAGE CONTAINER FOR ELECTRIC ORAL-HYGIENE DEVICE

(71) Applicant: Braun GmbH, Kronberg (DE)

(72) Inventors: Daniel DIETZEL, Kelkheim (DE); Ansgar HUEBNER, Bad Soden (DE); Frank KRESSMANN, Eschborn (DE); Frank MUELLER, Oberursel (DE); Hansjoerg REICK, Steinbach (DE); Anke RENZ, Schwalbach (DE); Markus SABISCH, Waldems (DE); Fritz SCHUBERT, Bad Camberg (DE); Egle KIIVER, Frankfurt (DE)

(21) Appl. No.: 15/130,226

(22) Filed: Apr. 15, 2016

(30) Foreign Application Priority Data
Apr. 28, 2015 (EP) 15165390.4
Mar. 9, 2016 (EP) 16159500.4

(51) Int. Cl. A61C 17/22; A46B 17/06; A45D 44/18

(52) U.S. Cl. CPC A61C 17/224; A45D 44/18; A46B 17/065

(57) ABSTRACT

A storage container for an electric oral-hygiene device has a first casing part and a second casing part mechanically coupled to allow for an open-container state, in which the device can be removed from the container, and a closed-container state, in which the device is secured inside the container. The container further has a charging unit for selectively charging the device, an electrical input connector at an outer side of the first or second casing part for providing energy to the charging unit, and at least one electrical output connector at an outer side of the first or second casing part. The charging unit is arranged for selectively providing the energy received at the electrical input connector via the electrical output connector to an external device.

### FIELD OF THE INVENTION

The present invention is concerned with a storage container for an electric oral-hygiene device having a charging unit for charging an electric oral-hygiene device disposed in the container and an electrical input connector for providing energy to the charging unit.

### BACKGROUND OF THE INVENTION

It is known that an electric oral-hygiene device storage container can be equipped with a charging unit and an electrical input connector for providing energy to the charging unit. Document US 2014/150189 A1 generally discusses such an electric oral-hygiene device storage container.

It is an object of the present disclosure to provide an electric oral-hygiene device storage container that is improved over the known containers or that provides at least an alternate realization of such a container.

### SUMMARY OF THE INVENTION

In accordance with one aspect there is provided a storage container for an electric oral-hygiene device, comprising a first casing part, at least a second casing part, which first and second casing parts are mechanically coupled to allow for an open-container state in which the electric oral-hygiene device can be removed from the container and a closed-container state in which the electric oral-hygiene device is secured inside the container, wherein the container further has a charging unit for selectively charging the electric oral-hygiene device, an electrical input connector realized at an outer side of the first or second casing part for providing energy to the charging unit, and at least one electrical output connector realized at an outer side of the first or second casing part, wherein the charging unit is arranged for selectively providing the energy received at the electrical input connector via the electrical output connector to an external device.

In accordance with one aspect there is provided a kit comprising the storage container as described in the present disclosure and an energy supply unit having an electrical output connector that is connectable with the electrical input connector of the container, wherein the electrical output connector of the energy supply unit and the electrical input connector of the container are designed as non-standardized coupling partners.

### BRIEF DESCRIPTION OF THE DRAWINGS

The present disclosure will be further elucidated by a detailed description of example embodiments and with reference to figures.

FIG. 1 is a depiction of an exemplary storage container, in a closed state, for an electric oral-hygiene device;

FIG. 2 is a depiction of the storage container shown in FIG. 1, in an open state, without an electric oral-hygiene device stored in the container;

FIG. 3 is a depiction of an embodiment of an outer side wall of the storage container shown in FIGS. 1 and 2, wherein an electrical input connector and an electrical output connector are realized together with further optional elements;

FIG. 4 is a depiction of an embodiment of the storage container shown in FIG. 2, wherein an electric oral-hygiene device and a replacement head for the electric oral-hygiene device are disposed, in a stored position, in the storage container;

FIG. 5 is a depiction of an exemplary storage container having optional elements that may be realized in such a container;

FIG. 6 is a depiction of an exemplary kit comprising an exemplary storage container and an energy supply unit; and

FIG. 7 is a schematic depiction of various possible electric parts and connections therebetween that may be present in exemplary embodiments of the storage container of the disclosure.

### DETAILED DESCRIPTION OF THE INVENTION

In accordance with the present disclosure, a storage container (or simply “container) for an electric oral-hygiene device (or simply “device”) comprises at least one electrical output connector arranged on an outer side of the first or second part of the casing. This enables one to use the container not only for storing and charging the removable electric oral-hygiene device, but also for charging an external device, e.g., a smartphone, a tablet computer, a pocket lamp, a fitness tracker etc. This enablement enhances the versatility of the container. In addition, the container may be equipped to house further parts such as replacement heads for the electric oral-hygiene device, a dental floss reservoir etc. The container may in particular be conveniently used during travel.

During travel, while the electric oral-hygiene device is being charged, e.g., in a hotel room, an external device can also easily be connected with the electrical output connector of the container so that carrying along a further charging device for this external device can be dispensed with. It is to be understood that the term “electric oral-hygiene device” means complete devices, such as, e.g., an electric toothbrush, as well as handles of such devices, to which a replaceable head (or heads) can be attached. The electrical output connector may in particular be arranged as a standardized outlet (e.g., male or female connector or plug), e.g., as an USB or mini USB outlet or as a DC connector, e.g., 2.1 mm or 2.5 mm pin size. In some embodiments, the electrical output connector can be arranged as an inductive electrical output connector, e.g., the electrical output connector may then be designed as an inductive electrical energy transfer unit in accordance with the Qi standard—or the electrical output connector may be designed in accordance with the Energy Matters Alliance standard.

The container comprises a casing having a first casing part and a second casing part that are mechanically connected. The first and second casing parts can be connected, e.g., by at least one hinge. Alternatively or additionally, the first and second casing parts can be connected by at least one snap or other type of lock or fastening connection, so that the container can be brought into an open position, in which the electric oral-hygiene device can be disposed in the container or can be taken out of the container, and into a closed position, in which the electric oral-hygiene device is secured inside the container, e.g., during travel.

In some embodiments, at least one of the first or second casing parts comprises an inlay having a recess for at least accommodating the electric oral-hygiene device therein. The inlay may be arranged to be replaceable, e.g., for cleaning purposes or to adapt the container to differently shaped electric oral-hygiene device or devices (i.e., at least two different inlays can then be provided). The inlay may comprise further recesses for receiving other parts such as, e.g., a replacement head for use with the electric oral-hygiene device.

The container comprises an electrical input connector and a charging unit for selectively charging the electric oral-hygiene device. The charging unit may be arranged for enabling the charging function only in the closed state of the casing, but in some embodiments charging is also possible in the open state of the casing. While the electrical input connector may be arranged in any known or standardized manner (e.g., as a USB, mini USB or DC connector), the electrical input connector can in some embodiments be arranged as a non-standardized and/or proprietary connection. In the latter instance, the electrical input connector can be configured to be coupled only with a respective non-standardized and/or proprietary (complementary) coupling partner, so that only a defined connection, such as, e.g., a water-proof connection, can be established between the container and an energy source, e.g., a wall socket. In this way, regulatory and safety requirements can be met.

The charging unit may provide energy to the electric oral-hygiene device in an inductive manner—although a direct electrical connection is also contemplated. The container may comprise a pin disposed in a complementary recess in the electric oral-hygiene device, which pin may serve for positioning the electric oral-hygiene device correctly with respect to an inductive energy transmitter (e.g., a coil) of the charging unit.

A kit can comprise the container and an energy supply unit having an electrical output connector that is connectable to the electrical input connector of the container, as is described herein. The electrical safety barrier for usage in a wet environment can then be implemented in the energy supply unit, which transforms the high AC voltage provided by a wall socket into a lower (e.g., 3 to 12 V) DC voltage.

In some embodiments, the charging unit comprises a charging prioritization unit for selectively controlling the supply of energy provided via the electrical input connector to the charging unit and via the charging unit then selectively to the electrical output connector. The charging prioritization unit may be arranged to apply pre-programmed (or hard-wired) rules for selectively providing energy for charging of the electric oral-hygiene device and for charging of an external device connected to the electrical output connector. Optionally energy may also be provided to further units, e.g., a sanitizing unit, a feedback element, or a powered fan (which units are described in more detail further below).

As one rule applied by the charging prioritization unit, energy supplied via the electrical input connector can be directly routed to the electrical output connector if the electric oral-hygiene device has been removed from the container or if the electric oral-hygiene device is already fully or sufficiently charged (i.e., selection in dependence on the charging state of the electric oral-hygiene device). An external device connected with the electrical output connector can then be provided with electrical energy, e.g., for the purpose of charging the external device. A further rule that may be applied by the charging prioritization unit is the following: if the electric oral-hygiene device is still to be charged, energy supply to the electrical output connector may be inhibited or may be controlled to be intermittent so that charging of the electric oral-hygiene device is prioritized.

In some embodiments, the container comprises a user-input unit, e.g., a switch or a wireless receiver unit for receiving signals from an external device, by which a user can set a prioritization of the charging unit or can override a charging prioritization unit if such is present. The user-input unit may even be arranged such that the user can reprogram the charging prioritization unit, e.g., the user-input unit may be a Bluetooth receiver that can receive commands from a computer having a Bluetooth transmitter. By such a user-input unit it may be achieved that energy is directly provided to the electrical output connector, instead of to the charging unit, for charging of the electric oral-hygiene device to thus manually prioritize the electrical output connector (and thus, e.g., prioritize charging of an external device over charging of the electric oral-hygiene device).

In some embodiments, the container comprises an energy source, e.g., a rechargeable accumulator or a replaceable energy source such as a battery. The charging unit (which may be controlled by the charging prioritization unit) may use the energy source to charge the electric oral-hygiene device and/or an external device via the electrical output connector (in particular in cases when no energy is provided via the electrical input connector). In case of a rechargeable energy source, the charging unit may charge the energy source when it is not fully charged, e.g., when the electric oral-hygiene device is already fully charged. The charging prioritization unit, if present, may take over the selective control over the charging sequence.

In some embodiments, the container further comprises at least one feedback element for indicating, e.g., a status of the charging unit. The status information may comprise at least one of a “no activity” feedback, a “charging of device ongoing” feedback, a “charging of device completed” feedback, a “energy provided at electrical output connector” feedback, a “charging of internal accumulator ongoing” feedback, an “internal accumulator is discharged” feedback, and the like. One skilled in the art would realize that the available feedback indications will depend on the configuration of the container; if, e.g., no rechargeable accumulator is present, then the feedback possibilities do not include a feedback related to the rechargeable accumulator.

The feedback element may be arranged for at least one of an audible, visual, or tactile feedback. The feedback element may be a light emitting element such as an LED, e.g., optionally a multi-color LED; a sound emission element, such as, e.g., a speaker and/or a motor-based sound generator; or a vibrator element, e.g., a piezo-vibrator. The feedback element may be arranged on an outer side (or outer surface) of the container, in particular if it is a visual feedback element. In some embodiments, the feedback element can be arranged at the removable electric oral-hygiene device, which can be used to take over the feedback function, e.g., an LED provided at the (visible) housing of the electric oral-hygiene device may be used for visual feedback—or a motor of the electric oral-hygiene device may be used for tactile and/or audible feedback.

In some embodiments, the container comprises a sanitizing unit arranged for sanitizing at least a part of the electric oral-hygiene device, e.g., a functional head of the electric oral-hygiene device, such as a brush head. The functional head may be attachable to and detachable from the handle of the device; and the container can be structured and configured to receive the functional head detached from the handle. For example, the sanitizing unit may comprise an ejector unit (e.g., a piezo-element-driven ejector nozzle) for ejecting a small amount of a sanitizing fluid (e.g., in the form of a fine spray) onto at least the part of the device to be sanitized when the container is closed. Alternatively or additionally, the sanitizing unit may comprise a UV light source for emission of germicidal ultraviolet radiation towards at least the part of the electric oral-hygiene device to be sanitized.

In some embodiments, the container can be arranged so that a display device is supported by the container when the container is in the open position. The container can then be equipped with at least one receiving structure for holding the display device, e.g., in an upright or slightly inclined position. The at least one receiving structure may in particular be arranged to hold in place a display device of a particular geometry. In some embodiments, at least two receiving structures are present, which may comprise a cut-out provided in the first casing part and a recess in the second casing part, wherein the recess may in particular be provided in a replaceable inlay so that the recess can be adapted to the size of the display device to be received. The display device may comprise, e.g., a smartphone.

In some embodiments, the container may include a humidity-removal system. The humidity-removal system may be active and/or passive. In some embodiments, the humidity-removal system comprises a reservoir having a moisture-absorbing material. The reservoir may be replaceable, e.g., the reservoir may be snap-connected to the container. Alternatively or additionally, the reservoir may be arranged so that the moisture-absorbing material can be replaced. The moisture-absorbing material may be a silica gel, but other known moisture-absorbing materials are can be used as well. Additionally or alternatively, the humidity-removal system may comprise an active and/or passive venting system, e.g., at least one venting opening and/or a powered fan.

In some embodiments, the container can be equipped with at least one replaceable inlay. E.g., at least one of the casing parts may be arranged to have a generally shell-like structure in which an inlay may be snap-fitted or press-fitted. Such an inlay may be adapted to a particular electric oral-hygiene device. The inlay may comprise deformable foam material to allow adaptation to differently shaped electric oral-hygiene devices.

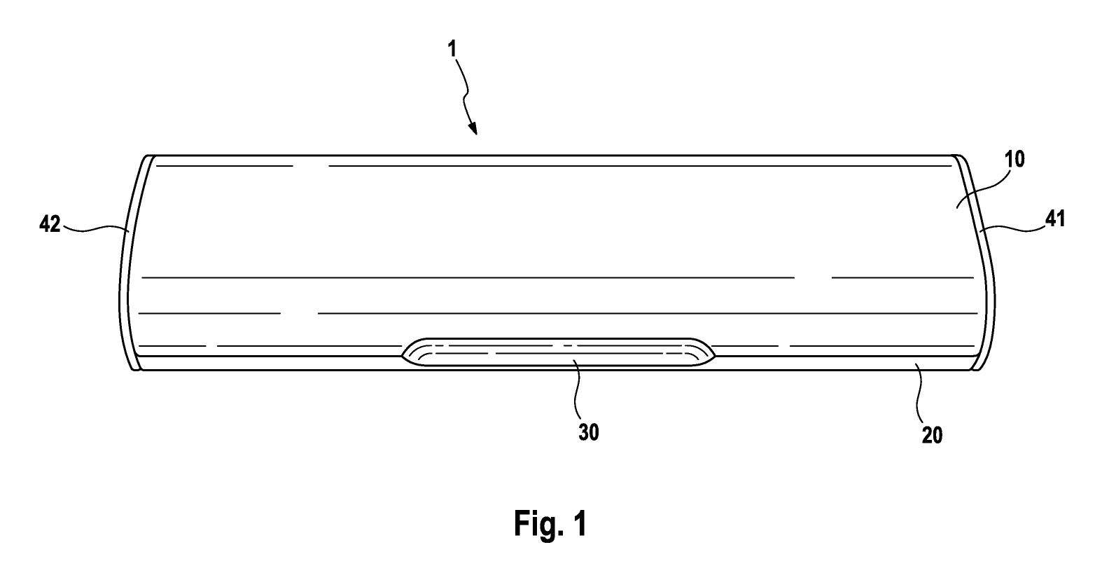

FIG. 1 is a depiction of an example container 1 in a closed state. The container 1 has a first casing part 10 and a second casing part 20. In the shown embodiment, an optional locking element 30 keeps the first and second casing parts 10, 20 together in the closed state and a user may need to press against the locking element 30 to release the locking function and to open the container 1. A spring element may be arranged to push the first and second casing parts 10, 20 apart once the locking function is released. The locking element 30 may be arranged at either one of the first and second casing parts 10, 20. In the shown embodiment, the container 1 has side walls 41, 42 that may be attached to or integrally connected with either one of the first or second casing parts 10, 20. Generally, a locking function may be achieved by any suitable means, e.g., by a snap hook, a magnetic lock, a hook and loop fastener, an adhesive connection, a press-fit connection etc.

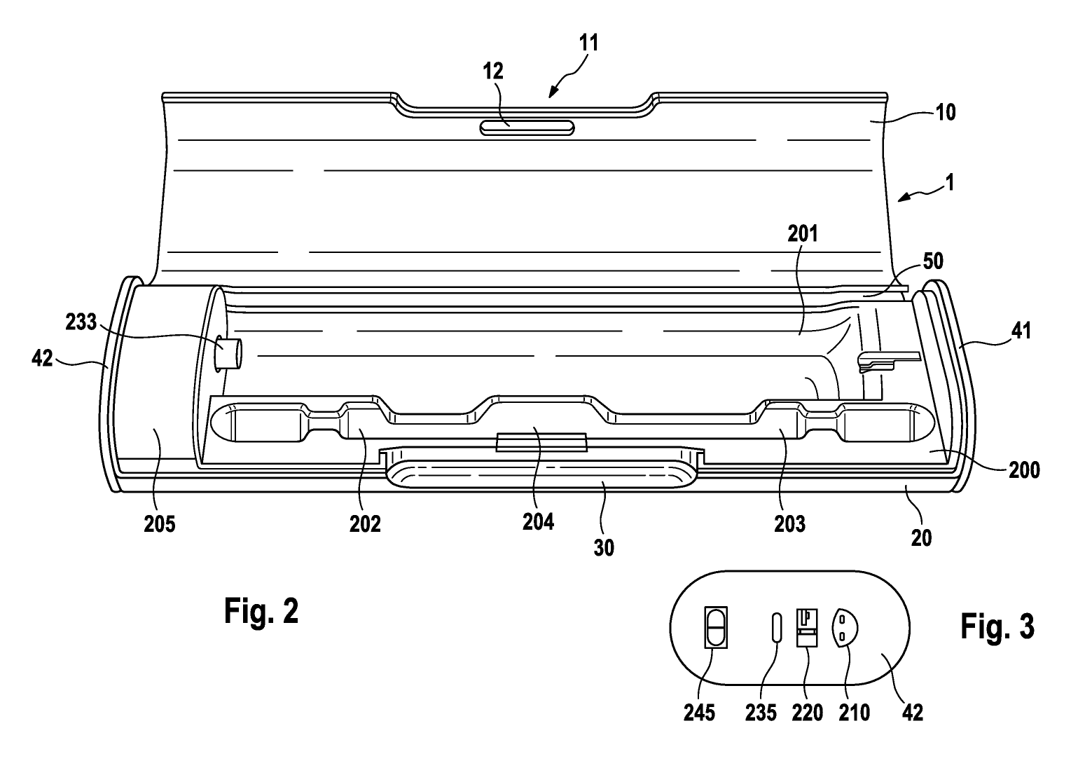

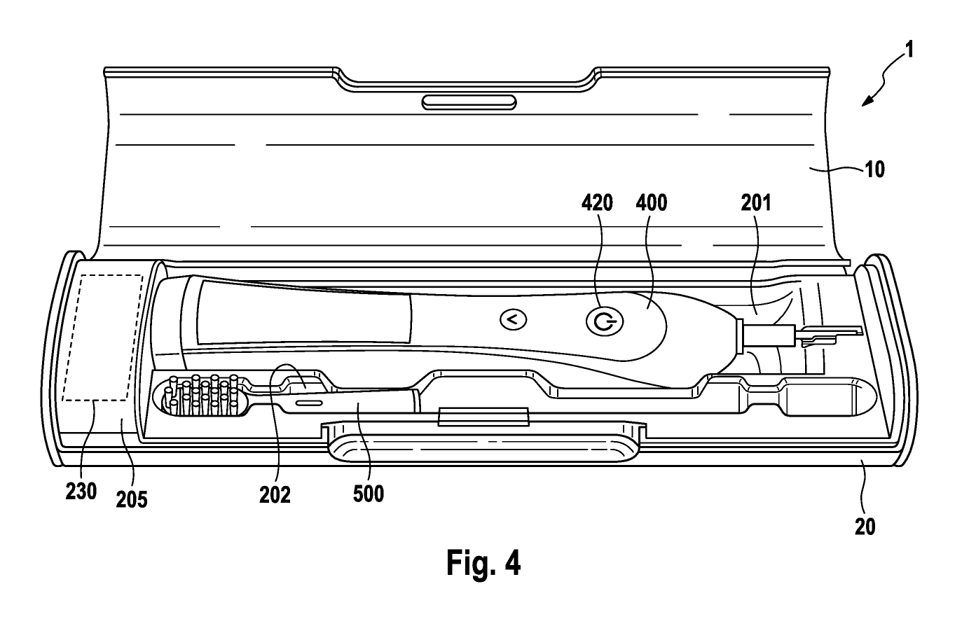

FIG. 2 is a depiction of a container 1 as shown in FIG. 1 but in an open state in which the first casing part 10 (taking the function of a lid or cover) is in a raised position. The removable electric oral-hygiene device is not shown in FIG. 2 for sake of visibility other parts of the container 1. FIG. 4 is a picture of the complete container with the removable electric oral hygiene disposed in the inner cavity of the container. The first casing part 10 may have a cut-out 11 that may serve to receive a display device (e.g., a smartphone) to keep the display device in an upright or inclined position. In the shown embodiment, a through-hole 12 is provided in the first casing part 10, which through-hole 12 may serve to receive a projection of the locking element 30. A strip 50 of thin material serves in the shown embodiment as hinge (here: a film hinge) arranged between the first casing part 10 and the second casing part 20. Any other type of hinge is also contemplated, e.g., a living hinge, a pivot hinge, a friction hinge, a self-closing hinge etc.

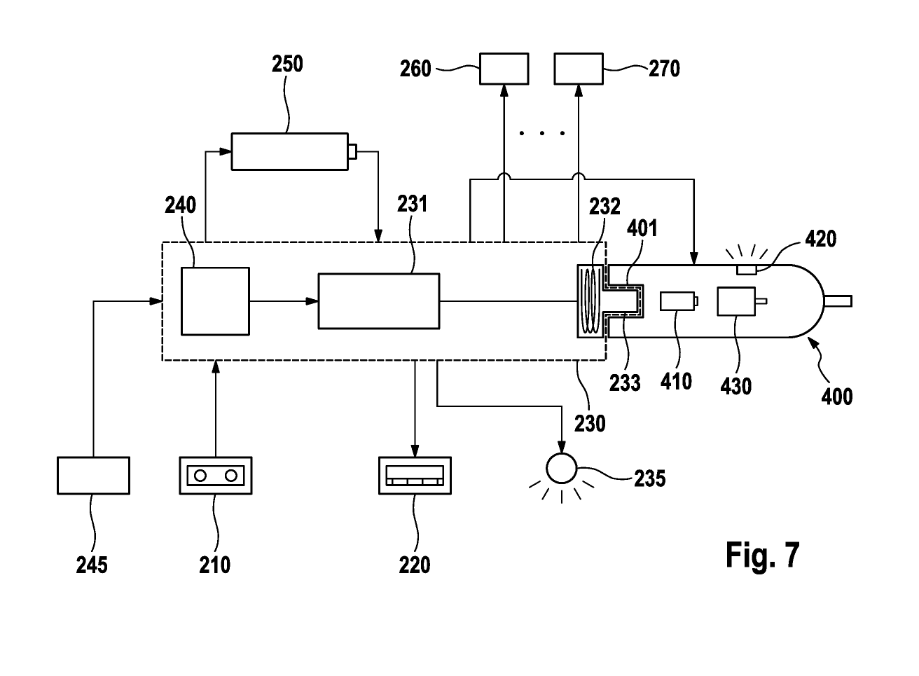

The second casing part 20 serves in the shown embodiment as storage portion of the container 1. The second casing part 20 together with the side walls 41 and 42 forms a shell-like tray. An inlay 200 is inserted into this tray. The inlay 200 is in particular a replaceable inlay so that it can be cleaned or replaced by another inlay to adapt the container to receive other items or to replace a soiled inlay by a fresh inlay. The inlay 200 has four depressions 201, 202, 203, 204 of which depressions 202, 203, and 204 are interconnected. Generally, the inlay 200 may have at least one depression, but any other number of depressions is possible as well. A depression 201 is shaped for receiving the removable electric oral-hygiene device (see FIG. 4). The three interconnected depressions 202, 203, and 204 are shaped to receive replacement heads for the electric oral-hygiene device (see FIG. 4) in the outer depressions 202 and 203. The center portion 204 of the interconnected depression is shaped so that it can receive a display device, which would then be supported by the center portion 204 of the interconnected depression and optionally also by the cutout 11 in the first casing part. On the left-hand side of the depicted container example, a box 205 fills the container, which box 205 in the shown embodiment is arranged to house a charging unit and optionally at least one further electric part as will be explained in the following. In some embodiments, a charging unit is housed in free space under the inlay 200 or in the first casing part 10 etc. A pin 233 extends from the box 205, which pin 233 is shaped so that it can be received in a recess of the electric oral-hygiene device to be received in the depression 201. The pin 233 then serves to hold the electric oral-hygiene device in an optimal position for inductive charging. While reference is made to a charging unit and optionally at least one further optional electric part that may be housed in box 205 in the following description referring to FIGS. 3 to 6, some example embodiments of the charging unit and the at least one optional further electric part of the container 1 are described in connection with FIG. 7 further below.

FIG. 3 is a depiction of the outer side of side wall 42 arranged on the left-hand side of the container (i.e. the side at which the box 205 housing the charging unit is arranged) shown in FIG. 2. The side wall 42 is part of the outer side of the container and is a portion of the second casing part 20. The side wall 42 has an electrical input connector 210, an electrical output connector 220, an optional feedback element 235 that may be realized as a light emission element, but which could alternatively or additionally be realized as an audible feedback element (e.g., a loudspeaker) or a tactile feedback element (e.g., a vibrator element), and an optional user-input unit 245 here realized as a switch for manually overriding a standard charging scheme as is explained in connection with FIG. 7 below. Generally, an electrical input connector and an electrical output connector (as well as the further optional elements) may each be arranged at any outer side of the container and FIG. 3 is just showing a particular example.

FIG. 4 is a depiction of the container shown in FIG. 2 with an electric oral-hygiene device 400 (here: a handle of an electric toothbrush) received in depression 201. The electric oral-hygiene device 400 may have an illuminated switch 420. By a dashed line it is indicated that box 205 can comprise a charging unit 230 that receives energy at least via an electrical input connector 210 as, e.g., shown in FIG. 3 for in particular charging a rechargeable battery provided in the electric oral-hygiene device 400 and which charging unit 230 is arranged to also selectively provide energy at the electrical output connector 220 (as, e.g., shown in FIG. 3). A replacement head 500 for the electric oral-hygiene device 400 is received in depression 202.

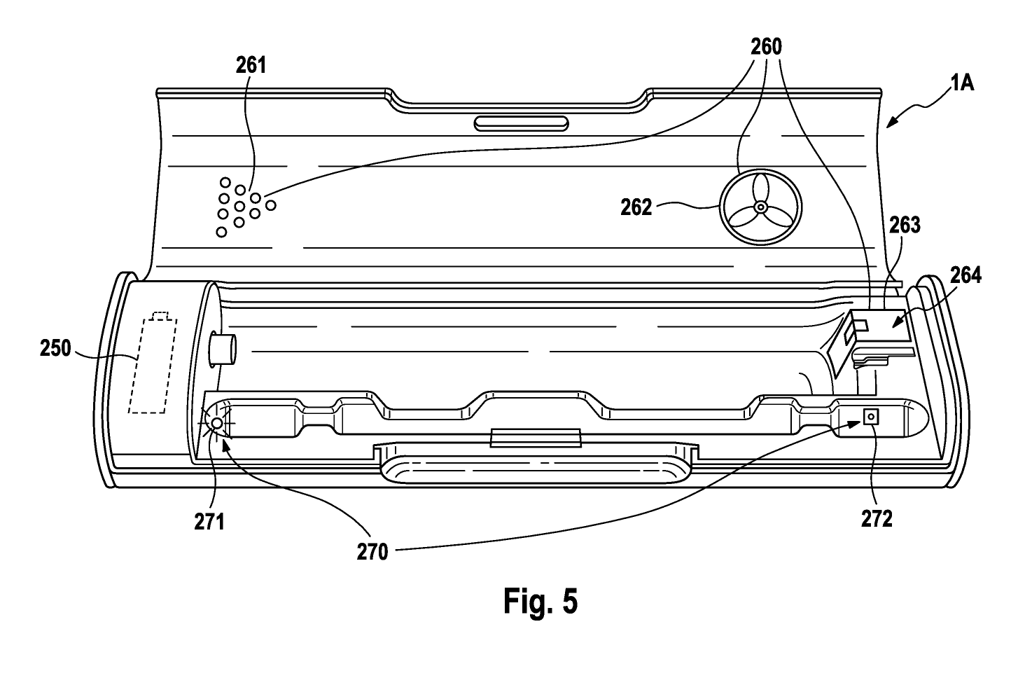

FIG. 5 is a depiction of an example container 1A having several optional features. It is indicated that the container 1A may comprise an accumulator (rechargeable battery)—drawn in dashed lines—for storing energy so that powered elements of the container 1A can be provided with energy even in circumstances where a connection to an external energy source (e.g., wall socket) is not present. It is shown by way of example that the container 1A may comprise a humidity-removal system 260 and/or a sanitizing unit 270. The humidity-removal system 260 may comprise at least one of the following: (1) at least one venting opening 261, (2) an actively driven venting unit 262—here realized as a fan, or (3) a reservoir 263 comprising humidity absorbing material 264. The sanitizing unit 270 may comprise at least one of the following: (1) a UV light emitting element 271 (e.g., an UV LED) or (2) a spray unit for generating a spray of sanitizing liquid (e.g., a piezo driven spray nozzle), which spray unit may comprise a reservoir of sanitizing liquid.

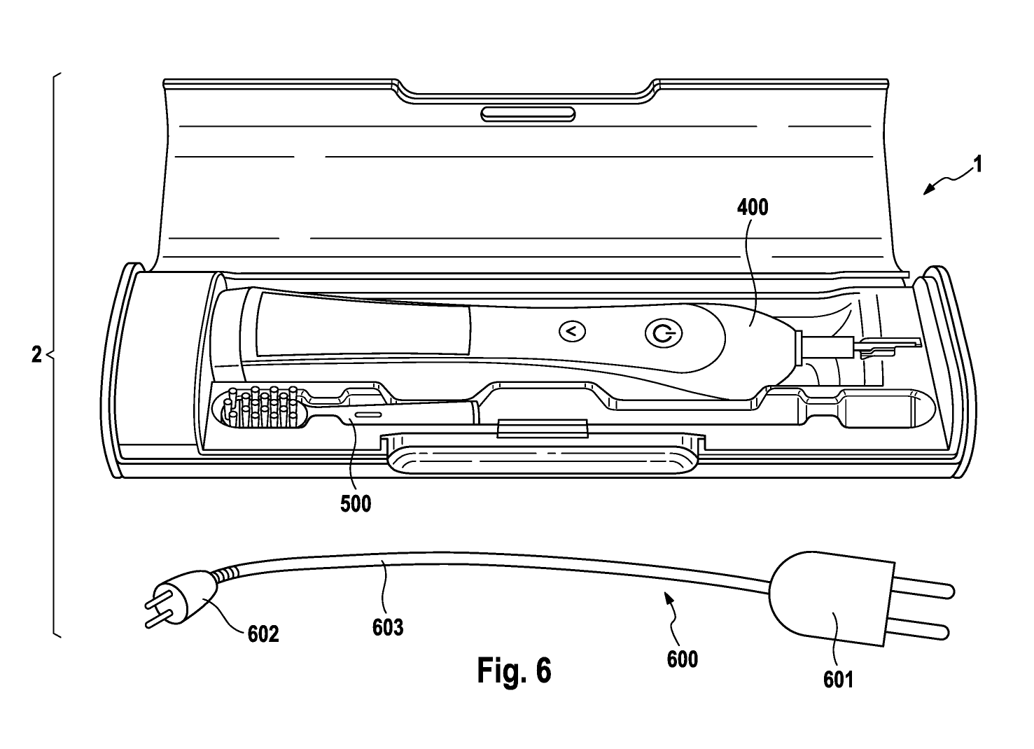

FIG. 6 is a depiction of a kit 2 comprising a container 1 as proposed housing an electric oral-hygiene device 400 (realized again as a handle) and a replacement head 500 for the electric oral-hygiene device 400 and an energy supply unit 600, which here has a energy plug 601 in which an AC-DC or an AC-AC transformer is included so that e.g., a 12 Volt DC or AC voltage can be provided at the electrical input connector 210 (FIG. 3) of the container 1 via an electrical output connector 602 of the energy supply unit 600. In particular, the electrical output connector 602 of the energy supply unit 600 and the electrical input connector 210 of the container 1 are realized as complementary non-standard connectors to assure that only an energy supply unit 600 providing a water-proof connection can be connected to the container 1, which container 1 may typically be placed in the bathroom.

FIG. 7 is a schematic depiction of the possible electrical parts and electrical connections of a container as discussed herein. This depiction shows various possibilities that do not necessarily need to be present in all embodiments of a proposed container and arbitrary combinations of the optional features are to be considered. A proposed container comprises an electrical input connector 210, an electrical output connector 220 arranged on an outer side of the casing and a charging unit 230 for selectively charging an electric oral-hygiene device 400 that comprises a rechargeable energy source 410 such as a rechargeable battery (accumulator or secondary battery). The electrical input connector 210 is arranged for receiving energy from an energy source such as from a wall socket via, e.g., an AC-DC energy converter realizing an energy supply unit. The electrical input connector 210 is coupled with the charging unit 230 to provide the energy to the charging unit 230. The charging unit 230 is coupled with the electrical output connector 220 and is arranged to selectively provide energy at the electrical output connector 220.

In some embodiments the container comprises at least one further electric part connected with at least one of the previously mentioned parts such as a charging prioritization unit 240, a user-input unit 245, a feedback element 235, a rechargeable battery 250, a humidity-removal system 260 and/or a sanitizing unit 270 that may each be electrically coupled with the charging unit 230. A container as proposed may comprise one or several of these further electric parts and respective electric connections.

In some embodiments, the charging unit 230 comprises a charging circuit 231 and a charging coil 232 for wireless, inductive energy transfer to a charging coil of the electric oral-hygiene device 400, where the charging coil 232 may be arranged in a pin-like structure 233 adapted for sliding into a form-fitting recess 401 of the electric oral-hygiene device 400 so that the electric oral-hygiene device 400 has a defined position to the charging coil 232 during charging.

In some embodiments, the charging unit 230 comprises a charging prioritization unit 240 for prioritizing the provision of energy received via the electrical input connector 210 or alternatively or additionally drawn from a rechargeable energy source 250. The prioritization unit 240 may apply at least one of the following prioritization rules, where these rules may be present as original default settings or may be chosen via the user-input unit (which rules are given as examples—any other prioritization rule may be applied as well):
1. First charge the electric oral-hygiene device 400 until it is fully charged (or until it is charged to a predetermined degree, e.g., 80%) before energy is provided via the energy outlet 220 for charging an external device. The provision of energy via the electrical output connected is thus controlled in dependence on the charging state of the electrical oral-hygiene device.
2. Intermittently provide energy for charging of the electric oral-hygiene device 400 until it is fully charged (or until it is charged to a predetermined degree, e.g., 80%) with a first predetermined interval ratio, e.g., 50% or 80% and provide energy via the energy outlet 220 at a second predetermined interval ration of e.g., 50% (if the first predetermined interval ratio is 50%) or less than 50% (if the first predetermined interval ratio is 50%) and in this case use the remaining e.g., 10% for e.g., charging a rechargeable battery 250 (if such is present) or for energizing other loads of the container (e.g., a powered fan of a humidity-removal system 260 or a UV light source of a sanitizing unit 270, if one of such is present).
3. First provide energy received via the energy inlet 210 at the energy outlet 220 if a load is present at the energy outlet 220.
4. Draw energy from internal rechargeable battery 250 if no energy is provided via electrical input connector 210.

A user-input unit 245 may be provided for switching between prioritization rules, for overriding a prioritization rule by a user defined prioritization rule or for reprogramming the prioritization unit 240. The user-input unit 245 may thus be realized as a simple switch to switch between predefined prioritization rules or, e.g., as a Bluetooth based receiving device for wireless reprogramming of the prioritization unit 240 via an external device such as a smartphone (to which a respective reprogramming app has been loaded), tablet, or a computer.

The container may have a feedback element 235 for indicating, e.g., a charging state as has already been described. The feedback element 235 may be arranged at the outer side of the container. In some embodiments, the charging unit 230 is controllably coupled with the electric oral-hygiene device 400 so that the charging unit 230 can use a feedback element arranged at the removable electric oral-hygiene device 400, e.g., a light emission element 420 or a motor 430 of the electric oral-hygiene device 400 to provide visual, acoustic or tactile feedback.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A storage container for an electric oral-hygiene device, the storage container comprising:
a removable electric oral-hygiene device;
a first casing part;
at least a second casing part;
wherein the first and second casing parts are mechanically coupled to allow for an open-container state in which the electric oral-hygiene device can be removed from the container and a closed-container state in which the electric oral-hygiene device is secured inside the storage container;
wherein the container further has a charging unit for selectively charging the electric oral-hygiene device, an electrical input connector realized at an outer side of the first or second casing part for providing energy to the charging unit, and at least one electrical output connector realized at an outer side of the first or second casing part, wherein the charging unit is arranged for selectively providing the energy received at the electrical input connector via the electrical output connector to an external device.

2. The storage container of claim 1, wherein the electrical output connector is designed as a standardized connector such as an USB connector.

3. The storage container of claim 1, wherein the charging unit comprises a charging prioritization unit arranged to control the supply of energy provided via the electrical input connector to the charging unit and to the electrical output connector, wherein the charging prioritization unit is arranged to control the supply of energy in dependence on the charging state of the electric oral-hygiene device.

4. The storage container of claim 1, further comprising a user-input unit arranged for manually overriding the charging prioritization unit.

5. The storage container of claim 1, further comprising at least one feedback element for providing at least one of an audible feedback, visual feedback, and tactile feedback about at least one of a status of the charging unit and a charging process performed by the charging unit.

6. The storage container of claim 1, wherein the charging unit or the charging prioritization unit is arranged to use as the feedback element a feedback element arranged at the electric oral-hygiene device to provide the feedback.

7. The storage container of claim 1, further comprising an internal energy source.

8. The storage container of claim 1, wherein the charging unit is arranged to selectively charge the accumulator when energy is provided via the electrical input connector or to provide energy via the electrical output connector drawn from the accumulator.

9. The storage container of claim 1, further comprising a sanitizing unit for sanitizing at least a portion of the electric oral-hygiene device.

10. The storage container of claim 1, wherein the sanitizing unit comprises a UV light source.

11. The storage container of claim 1, wherein the storage container has at least one receiving structure for holding a removable display device such as a smart phone, in particular wherein at each of the first and second casing parts a receiving structure is arranged to hold the removable display device in the open position of the container.

12. The storage container of claim 1, wherein the receiving structures comprise a cut-out in the first casing part and a recess in the second casing part, in particular wherein the second casing part comprises an inlay in which the recess is provided.

13. The storage container of claim 1, further comprising humidity-control system selected from a group consisting of a humidity-removal system, an active venting system, a passive venting system, and any combination thereof.

14. The storage container of claim 1, further comprising at least one replaceable inlay for receiving at least the electric oral-hygiene device.

15. A kit comprising the storage container of claim 1 and an energy-supply unit having an electrical output connector that is connectable with the electrical input connector of the storage container, wherein the electrical output connector of the energy-supply unit and the inlet of the container are structured and configured as non-standardized coupling partners.

16. The storage container of claim 5, wherein the feedback element is arranged on an outer side of the container.

17. The storage container of claim 7, wherein the internal energy source is selected from the group consisting of a rechargeable accumulator, a replaceable battery, and any combination thereof.

18. The storage container of claim 11, wherein the removable display device comprises a smart phone.

19. The storage container of claim 11, wherein at each of the first and second casing parts the receiving structure is arranged to hold the removable display device in the open-container state.

20. The storage container of claim 12, wherein the second casing part comprises an inlay in which the recess is provided.

21. The storage container claim 13, wherein the humidity-control system comprises at least one of a venting slit and a powered fan.

22. The storage container claim 21, wherein the humidity-control system comprises a reservoir of moisture-absorbing material.

23. The storage container claim 22, wherein the reservoir is replaceable.

24. The storage container claim 22, wherein the moisture-absorbing material is replaceable.

25. A storage container for an electric oral-hygiene device, the storage container comprising:
a first casing part; and
at least a second casing part;
wherein the first and second casing parts are mechanically coupled to allow for an open-container state, in which the electric oral-hygiene device can be removed from the container, and a closed-container state in which the electric oral-hygiene device is secured inside the storage container;
wherein the container further has a charging unit for selectively charging the electric oral-hygiene device when the electric oral-care device is disposed in the storage container, an electrical input connector located at an outer side of at least one of the first casing part and the second casing part for providing energy to the charging unit, and at least one electrical output connector located at an outer side of at least one of the first casing part and the second casing part, wherein the charging unit is arranged for selectively providing the energy received at the electrical input connector via the electrical output connector to an external device.
